(19) United States
(12) Patent Application Publication
(10) Pub. No.: US 2021/0113208 A1
(43) Pub. Date: Apr. 22, 2021

(54) WOUND CLOSURE DEVICE

(71) Applicant: The General Hospital Corporation, Boston, MA (US)

(72) Inventors: Kartik Mangudi Varadarajan, Belmont, MA (US); Harry E. Rubash, Weston, MA (US)

(21) Appl. No.: 16/981,955

(22) PCT Filed: Mar. 21, 2019
(86) PCT No.: PCT/US2019/023466

§ 371 Date: Sep. 17, 2020

Related U.S. Application Data
(60) Provisional application No. 62/645,973, filed on Mar. 21, 2018.

(51) Int. Cl. A61B 17/08

(52) U.S. Cl. CPC A61B 17/085; A61B 2017/081; A61B 2017/086

(57) ABSTRACT

A wound closure device is provided. The wound closure device includes a first pad with an adhesive surface and a second pad with an adhesive surface. The wound closure device also includes a first tension adjusting pad with a first section, a first connecting element and a second connecting element. The first tension adjust pad is configured to move the first pad towards the second pad when the first tension adjusting pad is moved away from the first pad. The wound closure device also includes a second tension adjusting pad with a second section, a third connecting element and fourth connecting element. The second tension adjusting pad is configured to move the second pad towards the first pad when the second tension adjusting pad is moved away from the second pad. The wound closure device further includes a first cross bar for securing the first and third connecting elements.

### CROSS-REFERENCES TO RELATED APPLICATIONS

This application is based on, claims priority to, and incorporates herein by reference in its entirety U.S. Provisional Application Ser. No. 62/645,973 filed on Mar. 21, 2018 and entitled “Wound Closure Device.”

### STATEMENT REGARDING FEDERALLY SPONSORED RESEARCH

Not Applicable.

### BACKGROUND OF THE INVENTION

### 1. Field of the Invention

The invention relates to devices and methods for incision closure and bringing together edges of a wound.

### 2.Description of the Related Art

A variety of devices are available for closure of surgical incisions, wounds and lacerations. Surgical sutures involve the use of a needle with an appropriate length of thread, depending on the shape, size, and location of the wound. Wound closure strips involve a surgical tape which pulls the patient's skin on either side of the wound together, thereby closing the wound. Wound strips and sutures are difficult to care for and to manipulate by both the patient and the medical professional applying them. Wounds located at joints in the body are particularly difficult areas to apply a wound closure device due to the inherent mobility of the joints.

Referring now to FIGS. 1A and 1B, one type of wound-closure device 100, includes a first component and a second interlaced component, each having an adhesive-backed wound-edge pad 102, 104 for attachment to a patient's skin adjacent to the wound or incision to be closed. Each of the wound-edge pads 102,104 is attached to an opposing tension adjusting pad 106, 107 that also has an adhesive backing. The wound-edge pads 102, 104 are connected to the respective tension adjusting pads 106 by elongate connecting elements 108.

The device 100 is used to close a wound or incision by following five general steps. The first step includes removing or peeling-off an adhesive backing at the back of the device (creating the structure of FIG. 1B), facing the patient's skin, thereby exposing a portion of the adhesive on underside of wound-edge pads 102, 104. Peeling off the adhesive backing results in separation of the first and second interlaced component. The first and second interlaced components can then be individually manipulated by the user. The first interlaced component is composed of wound-edge pad 102 and attached tension adjusting pad 106, and the second interlaced component is composed of wound-edge pad 104 and the attached tension adjusting pad 107. The exposed portions of the adhesive wound-edge pads 102, 104 are then individually adhered onto the patient's skin on either side of the wound/incision. The second step includes removing an adhesive backing from wound-edge pads 102,104 to expose the remainder of the adhesive underside of the pads 102,104, which are then adhered to the skin. Referring to FIG. 1A, the third step includes the user lifting the tension adjusting pads 106, 107 away from the patient's skin, and gently removing adhesive backing from each pad 106, 107. Step three includes the user pulling on the tension adjusting pads to close the wound. While maintaining this tensile force, the user lays down the adjusting pads causing the adhesive on the elongate connective elements 108 to adhere to the top surface of wound-edge pads 102,104. Finally, step five includes the tension adjusting pads 106,107 being snapped-off from the elongate connective elements 108. At the completion of device application, the wound-edge pads 102,104 on either side of the wound or incision, are connected only via the elongate connecting elements 108 . The elongate connecting elements 108 are relatively inextensible, and tension within these elements serves to keep the wound closed. See also U.S. Pat. No. 6,329,564.

However, there is a need for an improved wound closure device that provides better manipulation and control of the device while closing wounds located around joints such as the knee, hip, shoulder, and elbow.

### SUMMARY OF THE INVENTION

The present invention provides a wound closure device for the closure of surgical incisions, wounds and lacerations on a patient.

In one aspect, the present disclosure provides a wound closure device. The wound closure device can comprise a first pad, a second pad, a first tension adjusting pad, a second tension adjusting pad, and a first crossbar. The first pad can have an adhesive disposed on a surface of the first pad. The second pad can have an adhesive disposed on a surface of the second pad. The first tension adjusting pad can include a first section, a first connecting element, and a second connecting element. The first connecting element and the second connecting element can extend away from the first section and can be coupled to the first pad. The first tension adjusting pad can be configured to move the first pad towards the second pad when the first tension adjusting pad is moved away from the first pad. The first connecting element can have an adhesive disposed on a surface of the first connecting element for securing the first connecting element to the second pad. The second connecting element can also have an adhesive disposed on a surface of the second connecting element for securing the second connecting element to the second pad. The second tension adjusting pad can include a second section, a third connecting element, and a fourth connecting element. The third connecting element and the fourth connecting element can each extend away from the second section and can be coupled to the second pad. The second tension adjusting pad can be configured to move the second pad towards the first pad when the second tension adjusting pad is moved away from the second pad. The third connecting element can have an adhesive disposed on a surface of the third connecting element for securing the third connecting element to the first pad. The fourth connecting element can have an adhesive disposed on a surface of the fourth connecting element for securing the fourth connecting element to the first pad. The first crossbar can have an adhesive disposed on a surface of the first crossbar for securing the first connecting element to the third connecting element.

The wound closure device can further comprise a second crossbar and a third crossbar. The second crossbar can have an adhesive disposed on a surface of the second crossbar for securing the second connecting element to the third connecting element. The third crossbar can have an adhesive disposed on a surface of the third crossbar for securing the second connecting element to the fourth connecting element.

In another aspect, the present disclosure provides a wound closure device can have a first pad, a second pad, a third pad, a fourth pad, and a first tension adjusting pad. The first pad can have an adhesive disposed on a surface of the first pad. The second pad can have an adhesive disposed on a surface of the second pad. The third pad can have an adhesive disposed on a surface of the third pad. The fourth pad can have an adhesive disposed on a surface of the fourth pad. The first tension adjusting pad can include a first section, a first connecting element, and a second connecting element. The first connecting element and the second connecting element can extend away from the first section. The first connecting element can be coupled to the third pad. The second connecting element can be coupled to the fourth pad. The first tension adjusting pad can be configured to move the third pad and the fourth pad towards the first pad and the second pad, when the first tension adjusting pad is moved away from the third pad and the fourth pad. The first connecting element can have an adhesive disposed on a surface of the first connecting element for securing the first connecting element to the first pad. The second connecting element can have an adhesive disposed on a surface of the second connecting element for securing the second connecting element to the second pad.

The wound closure device can further comprise a second tension adjusting pad. The second tension adjusting pad can include a second section, a third connecting element, and a fourth connecting element. The third connecting element can extend away from the second section and the fourth connecting element extending away from the second section. The third connecting element and the fourth connecting element can be coupled to the second pad. The second tension adjusting pad can be configured to move the second pad towards the third pad and the fourth pad when the second tension adjusting pad is moved away from the second pad. The third connecting element can have an adhesive disposed on a surface of the third connecting element for securing the third connecting element to the third pad. The fourth connecting element can have an adhesive disposed on a surface of the fourth connecting element for securing the fourth connecting element to the fourth pad. The second tension adjusting pad can also include a first crossbar, a second crossbar, and a third crossbar. The first crossbar can have an adhesive disposed on a surface of the first crossbar for securing the first connecting element to the third connecting element. The second crossbar can have an adhesive disposed on a surface of the second crossbar for securing the second connecting element to the third connecting element. The third crossbar can have an adhesive disposed on a surface of the third crossbar for securing the second connecting element to the fourth connecting element.

In another aspect, the present disclosure provides a wound closure device can have a first pad, a second pad, a first tension adjusting pad, and a flexible tab. The first pad can have an adhesive disposed on a surface of the first pad. The second pad can have an adhesive disposed on a surface of the second pad. The first tension adjusting pad can be coupled to the first pad. The first tension adjusting pad can be configured to move the first pad towards the second pad when the first tension adjusting pad is moved away from the first pad. The first tension adjusting pad can also have an adhesive disposed on a surface of the first tension adjusting pad for securing the first tension adjusting pad to the second pad. The flexible tab can be coupled to the first and the second pad. The flexible tab can be free of an adhesive.

The wound closure device can further comprise a longitudinal dimension of the flexible tab positioned on an end of the first pad and a longitudinal dimension of the flexible tab is positioned on an end of the second pad. A second flexible tab can be coupled to the first and the second pad. The second flexible tab can be free of an adhesive. A third flexible tab can be coupled to the first and second pad. The third flexible tab can be free of an adhesive. The flexible tab and the second flexible tab do not overlap. The flexible tab and the second flexible tab can be substantially parallel to each other. The flexible tab and the second flexible can comprise a polymeric material. The polymeric material can comprise a plastic. The flexible tab can be located below the first pad, the second pad, and the first tension adjusting pad. The flexible tab can be substantially perpendicular to a wound when the first pad and the second pad are positioned on opposite sides of the wound. The wound closure device can also comprise a second tension adjusting pad coupled to the second pad. The second tension adjusting pad can be configured to move the second pad towards the first pad when the second tension adjusting pad is moved away the second pad. The second tension adjusting pad can have an adhesive disposed on a surface of the second tension adjusting pad for securing the second tension adjusting pad to the first pad.

In another aspect, the present disclosure provides a wound closure device can have a first pad, a second pad, a third pad, a fourth pad, a first tension adjusting pad, a second tension adjusting pad, and connecter. The first pad can have an adhesive disposed on a surface of the first pad. The second pad can have an adhesive disposed on a surface of the second pad. The third pad can have an adhesive disposed on a surface of the first pad. The fourth pad can have an adhesive disposed on a surface of the second pad. The first tension adjusting pad can be coupled to the first pad and also can be configured to move the first pad towards the second pad when the first tension adjusting pad is moved away the first pad. The first tension adjusting pad can have an adhesive disposed on a surface of the first tension adjusting pad for securing the first tension adjusting pad to the second pad. The second tension adjusting pad can be coupled to the third pad. The second tension adjusting pad can be configured to move the third pad towards the fourth pad when the second tension adjusting pad is moved away the third pad. The second tension adjusting pad can have an adhesive disposed on a surface of the second tension adjusting pad for securing the second tension adjusting pad to the fourth pad. The connector can be coupled to the first and the third pad. The connector can comprise a flexible material. The connector can allow parallel movement of the first and the third pad along axis defined by a wound. The connector fixes and prevents movement of the first pad relative to the third pad.

The wound closure can further include a longitudinal axis of the flexible tab. The longitudinal axis of the flexible tab can be substantially parallel to the wound when the first pad and the second pad are positioned on opposite sides of the wound and when the third pad and the fourth pad are positioned on opposite sides of the wound. A second connector can be coupled to the second and the third pad. A third connector coupled to the first and the second pad. The connector and the third connector do not overlap. The connector can be coupled to peripheral edges of the first pad and the third pad. The connector can further be coupled to the second pad and the fourth pad. The connector can also be coupled to peripheral edges of the first pad, the second pad, the third pad, and the fourth pad. The connector does not contact the adhesive surface of the first pad, the second pad, the third pad, or the fourth pad. A second connector can be coupled to the second pad and the fourth pad. The second connector can fix and prevents movement of the second pad relative to the fourth pad.

In another aspect, the present disclosure provides a wound closure device can have a first pad, a second pad, and a first tension adjusting pad. The first pad can have an adhesive disposed on a surface of the first pad. The first pad can have a line of material weakness. The second pad can have an adhesive disposed on a surface of the second pad. The first tension adjusting pad can be coupled to the first pad, and can be configured to move the first pad towards the second pad when the first tension adjusting pad is moved away the first pad. The first tension adjusting pad can have an adhesive disposed on a surface of the first tension adjusting pad for securing the first tension adjusting pad to the second pad. The line of material weakness can be a slit on an outer edge of the first pad. The slits can elongate the first pad in a direction substantially perpendicular to the first tension adjusting pad. The line of material weakness can also be a plurality of perforations extending across the first pad. The line of material weakness can be a plurality of perforations extending across the second pad.

The wound closure device can also include a second tension adjusting pad coupled to the second pad. The second tension adjusting pad can be configured to move the second pad towards the first pad when the second tension adjusting pad is moved away the second pad. The second tension adjusting pad can have an adhesive disposed on a surface of the second tension adjusting pad for securing the second tension adjusting pad to the first pad.

In another aspect, the present disclosure provides a wound closure device can have a first pad, a second pad, and a first tension adjusting pad. The first pad can have an adhesive disposed on a surface of the first pad. The first pad can include a line of material weakness at an outer edge of the first pad extending the first pad in a direction substantially perpendicular to the first tension adjusting pad. The line of material weakness can be a slit or a perforation. The second pad can have an adhesive disposed on a surface of the second pad. The first tension adjusting pad can be coupled to the first pad and can also be configured to move the first pad towards the second pad when the first tension adjusting pad is moved away the first pad. The first tension adjusting pad can have an adhesive disposed on a surface of the first tension adjusting pad for securing the first tension adjusting pad to the second pad. The first tension adjusting pad can include a first section, a first connecting element, a second connecting element, and a first crossbar. The first connecting element can extend away from the first section of the first tension adjusting pad. The first connecting element can be coupled to the first pad and can also have an adhesive disposed on a surface of the first connecting element for securing the first connecting element to the second pad. The second connecting element can extend away from the first section of the first tension adjusting pad, and can be coupled to the first pad. The second connecting element can have an adhesive disposed on a surface of the first connecting element for securing the second connecting element to the second pad. The first crossbar can have an adhesive disposed on a surface of the first crossbar connecting the first connecting element to the second connecting element. The first section can be removably coupled to the first tension adjusting pad at a line of material weakness. The line of material weakness can be a perforation. The first section can be separated from the first tension adjusting pad at the line of material weakness. The first connecting element and the second connecting element can both be rigidly flexible.

The wound closure device can also include a second tension adjusting pad. The second tension adjusting pad can be coupled to the second pad, and can be configured to move the second pad towards the first pad when the second tension adjusting pad is moved away the second pad. The second tension adjusting pad can have an adhesive disposed on a surface of the second tension adjusting pad for securing the second tension adjusting pad to the first pad. The second tension adjusting pad can also include a second section, a third connecting element, a fourth connecting element, and a second crossbar. The third connecting element can extend away from the second section of the second tension adjusting pad. The third connecting element can be coupled to the second pad. The third connecting element can have an adhesive disposed on a surface of the third connecting element for securing the third connecting element to the first pad. The fourth connecting element can extend away from the second section of the second tension adjusting pad, and can be coupled to the second pad. The fourth connecting element can have an adhesive disposed on a surface of the fourth connecting element for securing the fourth connecting element to the first pad. The second crossbar can have an adhesive disposed on a surface of the second crossbar connecting the third connecting element to the fourth connecting element. The second section of the second tension adjusting pad can be removably coupled to the second tension adjusting pad at a line of material weakness. The third connecting element and the fourth connecting element can be rigidly flexible.

In another aspect, the present disclosure provides a wound closure device can have a first pad, a second pad, a third pad, and a first tension adjusting pad. The first pad can have an adhesive disposed on a surface of the first pad. The first pad can include a line of material weakness at an outer edge of the first pad extending the first pad in a direction substantially perpendicular to the first tension adjusting pad. The line of material weakness can be a slit or a perforation. The second pad can have an adhesive disposed on a surface of the second pad. The third pad can have an adhesive disposed on a surface of the third pad. The first tension adjusting pad can be coupled to the first pad and can also be configured to move the first pad towards the second pad when the first tension adjusting pad is moved away the first pad. The first tension adjusting pad can have an adhesive disposed on a surface of the first tension adjusting pad for securing the first tension adjusting pad to the second pad. The first tension adjusting pad can include a first section, a first connecting element, a second connecting element, and a first crossbar. The first connecting element can extend away from the first section of the first tension adjusting pad. The first connecting element can be coupled to the first pad and can also have an adhesive disposed on a surface of the first connecting element for securing the first connecting element to the second pad. The second connecting element can extend away from the first section of the first tension adjusting pad, and can be coupled to the third pad. The second connecting element can have an adhesive disposed on a surface of the first connecting element for securing the second connecting element to the second pad. The first crossbar can have an adhesive disposed on a surface of the first crossbar connecting the first connecting element to the second connecting element. The first section can be removably coupled to the first tension adjusting pad at a line of material weakness. The line of material weakness can be a perforation. The first section can be separated from the first tension adjusting pad at the line of material weakness. The first connecting element and the second connecting element can both be rigidly flexible.

The wound closure device can be configured so that the adhesive disposed on the surface of the first crossbar of the first connecting element is for securing the first crossbar to the second pad. The adhesive disposed on the surface of the first crossbar of the first connecting element may be used to secure the first crossbar to the second pad. The first connecting element and the second connecting element can be rigidly flexible. The wound closure device can include a line of material weakness, which may be a perforation, where the first section is removably coupled to the second pad. At the line of material weakness, the first section may be separated from the first tension adjusting pad.

The wound closure device may further include a fourth pad, a second section of the second tension adjusting pad, a third connecting element, a fourth connecting element, and a second crossbar. The fourth pad may have an adhesive disposed on a surface of the fourth pd. The third connecting element may extend away from the second section of the second tension adjusting pad so that the third connecting element is coupled to the second pad. The third connecting element can have an adhesive disposed on a surface of the third connecting element for securing the third connecting element to the third pad. The fourth connecting element can extend away from the second section of the second tension adjusting pad so that the fourth connecting element is coupled to the fourth pad. The fourth connecting element may have an adhesive disposed on a surface of the fourth connecting element to secure the fourth connecting element to the third pad. Likewise, the second crossbar can have an adhesive disposed on a surface of the second crossbar that connects the third connecting element to the fourth connecting element so that the adhesive secure the second crossbar to the third pad. The third connecting element and the fourth connecting element can be rigidly flexible. The wound closure device can include a line of material weakness where the second section of the second tension adjusting pad is removably coupled to the second tension adjusting pad.

A wound closure device of the present disclosure has a number of advantages. For example, the wound closure device has a structure that allows for better manipulation and/or control compared to prior wound closure devices.

One advantageous structure may include, but is not necessarily limited to, the addition of one or more strip-like non-adhesive extensions extending from two adhesive wound pads, thereby joining the wound pads, to prevent separation of the first and second interlaced components following removal of an adhesive backing during a first step of the device use. The strip-like non-adhesive extensions can be placed at any locations on or beneath the wound pads.

Another advantageous structure may include, but is not necessarily limited to, one or more features that allow the wound closure device to stretch or elongate with the skin. The structure may comprise the addition of any number of slits or perforations on the wound pads.

Another advantageous structure may include, but is not necessarily limited to, one or more features that allow the wound closure device to stretch or elongate parallel to a wound line. The features may be reduced thickness zones between portions of the wound pads or between consecutive sets of wound pads. The two wound pad configuration may be further composed of, and amplified by, multiple consecutive wound pad configurations or sets wherein a film on the top of the wound pads is removed after the wound pads adhere to either side of the wound thereby permitting adjacent sets of the wound pads to move independent of each other in response to elongation or stretching of skin parallel to the wound line.

Another advantageous structure may include, but is not necessarily limited to, one or more features that allow the wound closure device to stretch or elongate both parallel and perpendicular to a wound line. The features may comprise the addition of cross-bars between adjacent elongate connecting elements such that the device can apply a compressive force across the wound whenever flexion or stretching parallel to the wound takes place. The wound pads may be further composed of multiple configurations, segments, or sets to facilitate stretching of the wound closure device parallel to the wound.

These and other features, aspects, and advantages of the present invention will become better understood upon consideration of the following detailed description, drawings, and appended claims.

### BRIEF DESCRIPTION OF THE DRAWINGS

FIG. 1A shows a perspective view of a prior art wound closure device.

FIG. 1B shows a perspective view of a prior art wound closure device.

FIG. 2 shows a perspective view of a pre-aligned wound closure device, according to one embodiment of the present disclosure.

FIG. 3A shows a top view of a wound closure device according to another embodiment of the present disclosure.

FIG. 3B shows a top view of the wound closure device of FIG. 3A in a stretched position.

FIG. 4A shows a top view of a wound closure device with addition of perforations on the pads according to another embodiment of the present disclosure.

FIG. 4B shows a top view of a wound closure device with reduced thickness zone(s) between consecutive wound-edge pads to facilitate device elongation parallel to wound line according to another embodiment of the present disclosure.

FIG. 5A shows a top view of a wound closure device with consecutive sets of pads, with a film adhering to the top of the pads according the another embodiment of the present disclosure.

FIG. 5B shows a top view of a wound closure device of FIG. 5A with the film removed after adhering the pads to either side of the wound, permitting adjacent sets of pads to move independent of each other in response to elongation/stretching of skin parallel to the wound.

FIG. 6A is a top view of a wound closure device designed such that at the completion of the wound closure device application, an alternating connection is formed between the elongate connecting elements via the use of crossbars according to another embodiment of the present disclosure.

FIG. 6B is a perspective view of a wound closure device of FIG. 6A stretching parallel to the wound line results in generation of compressive force at the wound line.

FIG. 7A is a top view of one step of a sequence of steps resulting in the formation of the alternating connection pattern between the elongate connecting elements of the wound closure device of FIG. 6A.

FIG. 7B is a top view of another step of a sequence of steps resulting in the formation of the alternating connection pattern between the elongate connecting elements of the wound closure device of FIG. 6A.

FIG. 8A is a top view of a wound closure device with alternating connection pattern between the elongate connecting elements at the cross-bars according to another embodiment of the present disclosure.

FIG. 8B is a top view of a wound closure device of FIG. 8A with sections of the tension adjusting pads removed.

FIG. 9A is a top view of a wound closure device with the wound-edge pads according to another embodiment of the present disclosure.

FIG. 9B is a top view of a wound closure device of FIG. 8 with segmented wound-edge pads according to another embodiment of the present disclosure.

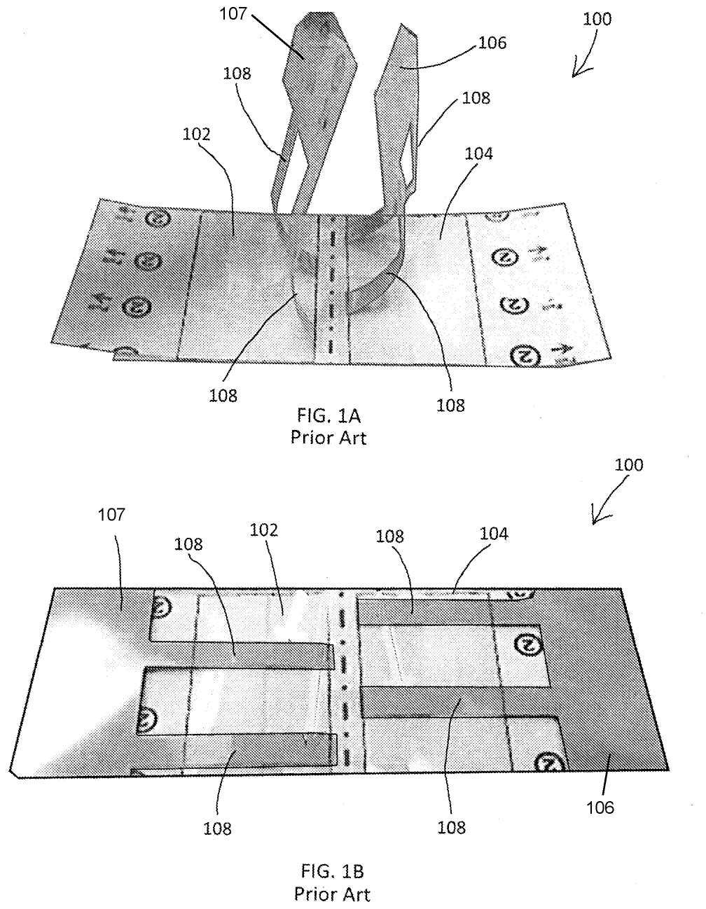

Like reference numerals will be used to refer to like parts from FIG. 1A to FIG. 9B in the following description of the drawings.

### DETAILED DESCRIPTION OF THE INVENTION

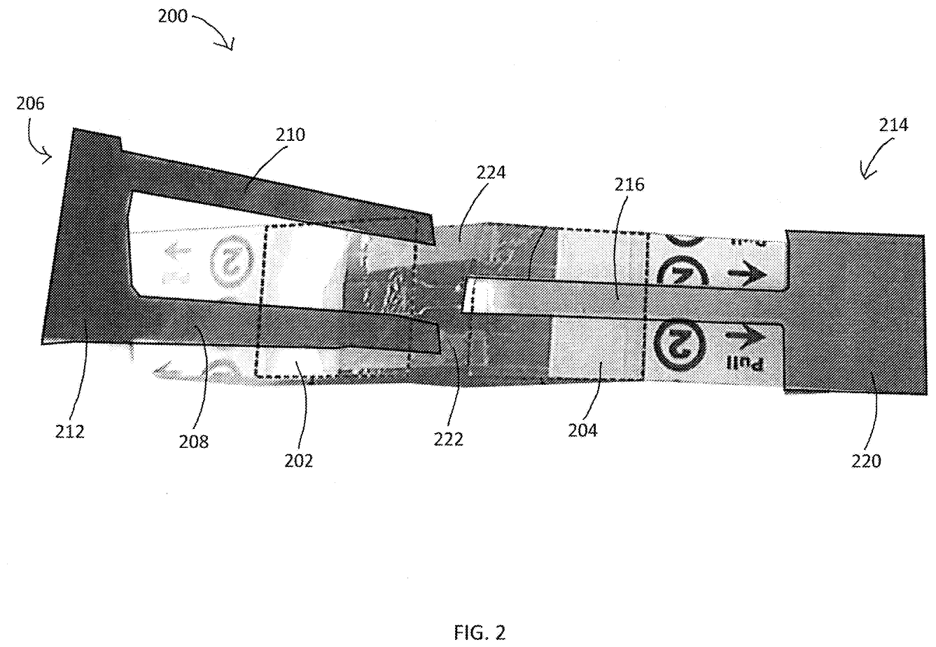

Referring now to FIG. 2, there is shown a wound closure device 200, according to one embodiment of the present disclosure. The wound closure device 200 includes a first pad 202 having an adhesive disposed on a surface of the first pad 202, and a second pad 204 having an adhesive disposed on a surface of the second pad 204. Emanating from the first pad 202 is a first tension adjusting pad 206 having elongated and generally parallel connecting elements 208, 210 integrally formed with a first section 212. Similarly, emanating from the second pad 204 is a second tension adjusting pad 214 having an elongated connecting element 216 integrally formed with a second section 220. In some embodiments, the second tension adjusting pad 214 can have additional connecting elements, which can be generally parallel to each other, and that can extend from the second pad 204 and can be integrally formed with the second section 220. As illustrated in FIG. 2, the connecting element 216 of the second tension pad 214 is received between the connecting elements 210, 208 of the first tension adjusting pad 206, although other configurations are possible.

The wound closure device 200 also includes a tab 222 that is coupled to the first pad 202 and the second pad 204, and that is free of any adhesive. The tab 222 is located on corresponding edges of the first pad 202 and the second pad 204.

The tab 222 can be located below the first and the second tension adjusting pads 206, 214, and can be located above the adhesive surfaces of the first and the second pads 202, 204. The tab 222 or additional tabs connecting the first and the second pads 202, 204 together, can be placed at any location on the first and the second pads 202, 204, such as beneath or adjacent to the connecting elements (e.g., connecting elements 208, 210, and 216) of the tension adjusting pads (e.g., the first and the second tension adjusting pads 206, 214). Shown in FIG. 2, is an additional tab 224, which is identical to the description and placement of the tab 222.

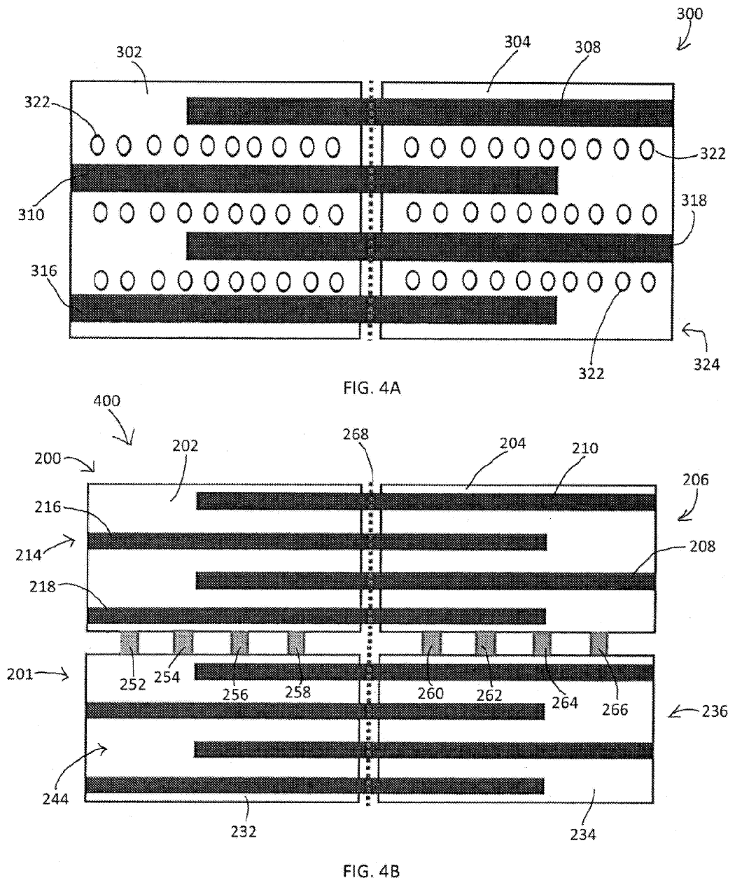

As detailed above, the prior art wound closure device of FIGS. 1A-1B has limitations. For example, when the adhesive backing is removed at step one, the first and second interlaced components (e.g., the first and the second pads 202, 204) can move away from each other in an unconstrained fashion, making the manipulation and control of the FIGS. 1A-1B device challenging. This is particularly true when attempting to close relatively small wounds or when using a small sized device. Therefore, in one embodiment (e.g., FIG. 2) the first and the second pads 202, 204 are joined by one or more strips/tabs of non-adhesive material (e.g., the tabs 222, 224), which constrain movement of the first and the second pads 204, 208, and thus prevents separation of the first and the second pads 204, 208 following removal of adhesive backing that protects the adhesive surfaces of the first and the second pads 204, 208. The addition of the tab 222, or additional tab(s) (e.g., tab 224), makes the handling of the device much easier. As another example of an advantage, when the tension adjusting pads 206, 214 are pulled to close the wound, the tab 222 or additional tab 224, which are flexible, move away from the skin, for example by bending/folding upwards, to allow for wound closure. Once the wound closure device 200 is fully applied and the wound closed, the tab 222 or additional tab 224, bend upwardly, and can be trimmed-off, such as with a scissors or other shearing devices known in the art. In some embodiments, the tabs 222, 224 can include a polymeric material, which can be a plastic. In other embodiments, the one or more tabs 222, 224 are substantially perpendicular to a longitudinal axis defined by a wound line (such as 268 in FIG. 4B), where substantially perpendicular to can deviate a number of degrees.

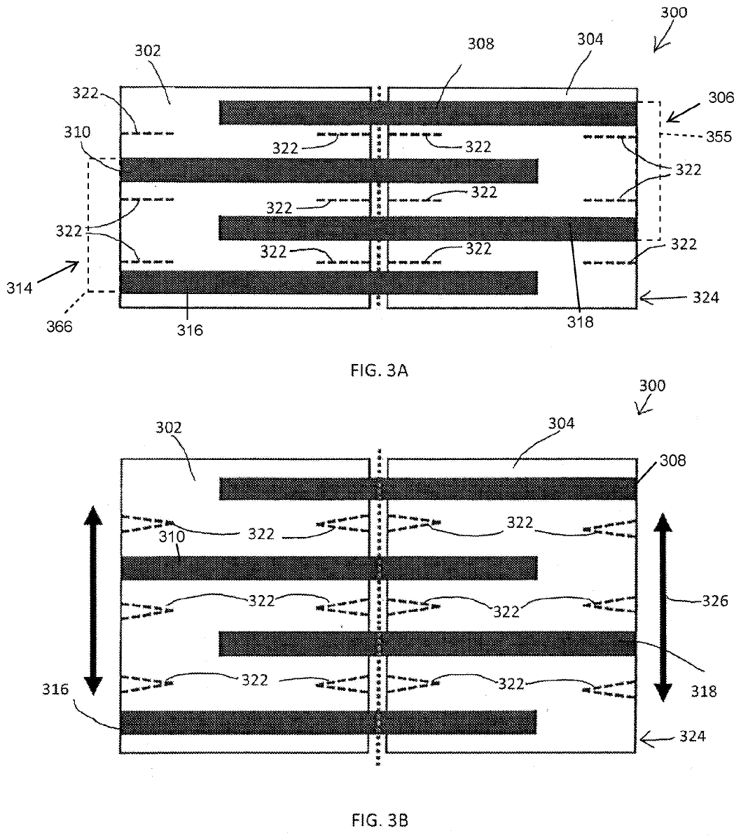

Referring now to FIGS. 3A and 3B, a wound closure device 300 according to another embodiment is shown. The wound closure device 300 has a first pad 302, a second pad 304, and a first tension adjusting pad 306. The first pad 302 can have an adhesive disposed on a surface of the first pad 302. The second pad 304 can have an adhesive disposed on a surface of the second pad 304. The adhesive allows the first pad 302 and the second pad 304 to stick to other surfaces of the wound closure device 300 as well as a patient whose wound is being closed. The first tension adjusting pad 306 can be coupled to the first pad 304, and configured to move the first pad 302 towards the second pad 304 when the first tension adjusting pad 306 is moved away the first pad 302. The first tension adjusting pad 306 can also have an adhesive disposed on a surface of the first tension adjusting pad 306 for securing the first tension adjusting pad 306 to the second pad 304. The first pad 302 can have a line of material weakness 322. The line of material weakness can be slits or a plurality of perforations. The slits can be located on an outer edge 324 of the first pad 302. The perforations can extend across the first pad 302, as shown in FIG. 4A, from the outer edge 304 of the first pad 302 to another opposite outer edge of the first pad 302. The slits and perforations can elongate the first pad 302 in a longitudinal direction 326 substantially perpendicular to the first tension adjusting pad 306. The first tension adjusting pad 306 includes connecting elements 308, 318 that are connected to a first section 355 that is removed during device application. Connecting elements 308, 318 are elongated and generally parallel to each other.

The wound closure device 300 can also have a second tension adjusting pad 314 coupled to the second pad 304. The second tension adjusting pad 314 can be configured to move the second pad 304 towards the first pad 302 when the second tension adjusting pad 314 is moved away the second pad 304. The second tension adjusting pad 314 can also have an adhesive disposed on a surface of the second tension adjusting pad 314 for securing the second tension adjusting pad 314 to the first pad 302. The second tension adjusting pad 314 includes connecting elements 310, 316 that are connected to a first section 366 that is removed during device application.

FIG. 4B shows a wound closure device 400, according to some embodiments of the disclosure. The wound closure device 400 includes sub components, for example, two or more wound closure devices 200, 201, which are similar or identical to the wound closure device 200 of FIG. 2, thus any discussion with regard to the wound disclosure device 201 also pertains to the wound disclosure device 201. The wound closure device 400 includes two wound closure devices 200, 201, as previously described. Thus, for explanatory purposes, the first closure device 400 includes a first wound closure device 200 having a first and second pads 202, 204, and the second closure device 201 includes a third and fourth pads, 232, 234. As illustrated in FIG. 4B, the wound closure device 200 includes a first and second tension adjusting pads 206, 214, as previously described above, although the second tension adjusting pad 214 includes a connecting element 218 emanating from the second pad 204 and integrally formed with the second section 220. As illustrated in FIG. 4B, the connecting element 216 of the second tension pad 214 is received between the connecting elements 210, 208 of the first tension adjusting pad 206, and the connecting element 208 is received between the connecting elements 216, 218 of the second tensioning pad 214. Shown also in FIG. 4B, each the corresponding section 212, 214 has been removed from the corresponding tension adjusting pad 206, 214, respectively (e.g., via a perforated line, or by shearing off the sections, after placement of the wound closure device 200 on the subject. As detailed above, the wound closure device 201 is identical to the wound closure device 200. Thus, similar components previously described above with regard to the wound closure device 200 also pertain to the wound closure device 201. For example, the wound closure device 201 has third and fourth pads 232, 234, which correspond to the first and second pads 202, 204 of the wound closure device 200. The wound closure device 201 also has a third and fourth tensioning pads 236, 244 corresponding to the first and the second tensioning pads 202, 204.

The wound closure device 400 includes one or more connectors (e.g., connectors 252, 254, 256, 258, 260, 262, 264, 266), which can couple various pads together (e.g., the first pad 202 to the third pad 232, etc.). As illustrated in FIG. 4B the wound closure system has eight connectors 252, 254, 256, 258, 260, 262, 264, 266, which are flexible. Four of the eight connectors, connectors 252, 254, 256, 258, couple the first pad 202 to the third pad 232 together, and the remaining four connectors, connectors 260, 262, 264, 266 couple the second pad 204 to the fourth pad 234. In some embodiments, and as illustrated in FIG. 4B, the connectors 252, 254, 256, 258, 260, 262, 264, 266 do not overlap.

When the wound closure device 400 (e.g., each of the wound closure devices 200, 201) are placed across a wound line 268, and installed as previously described, the longitudinal dimensions of each of the connectors 252, 254, 256, 258, 260, 262, 264, 266 are substantially parallel to the axis defined by the wound. Substantially parallel in this sense could be deviating 20 degrees, 10 degrees, 5 degrees, 1 degrees, any degree within this range, or other degrees. Also, when the wound closure device 400 is installed, the connectors 252, 254, 256, 258, 260, 262, 264, 266, which are flexible, allow the wound closure system to stretch or elongate parallel to the wound line 268 (e.g., during flexion/extension of a joint). Additionally, the use of reduced spacing between portions of the pads (e.g., between the first and the third pads 202, 232, etc.) can allow the wound closure device 400 to expand parallel to the wound edge 268. This can aid in comfort for the user, allowing the user to move, while simultaneously preventing reopening of the wound edge 268.

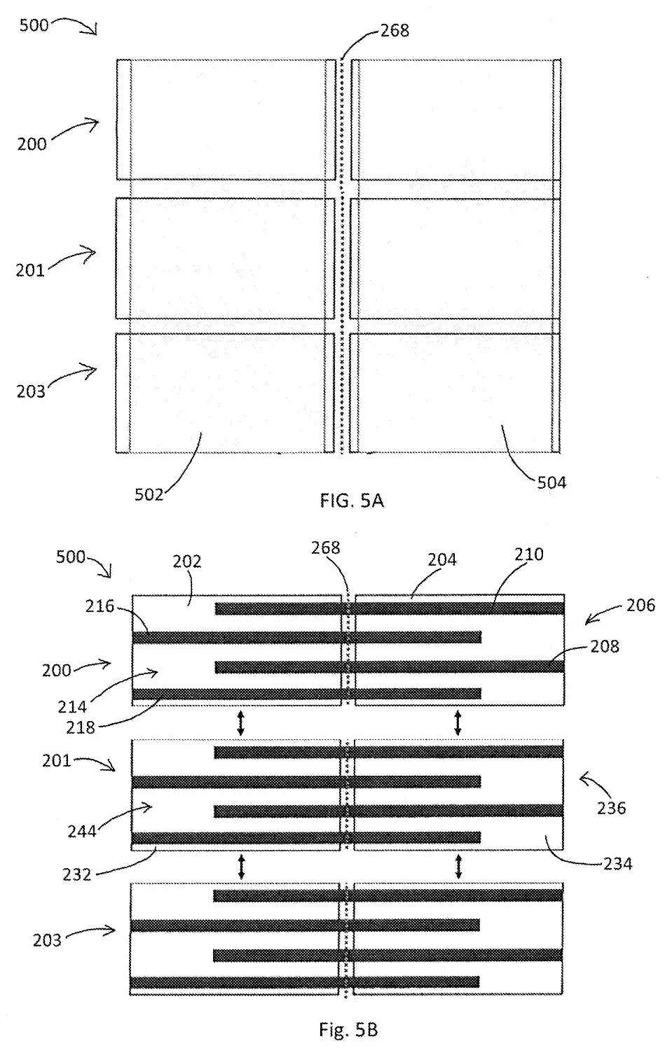

FIGS. 5A and 5B also show a wound closure device 500, according to some embodiments of the disclosure, with a slightly different configuration for the one or more connectors. Thus, any discussion with regard to the wound disclosure device 500 also pertains to the wound disclosure device 400. Additionally, the wound closure device 500 includes sub components, specifically wound closure devices 200, 201, and 203. The wound closure devices 200, 201 have been previously described and are identical to the wound closure device 203. Thus, the discussion above with regard to the wound closure devices 200, 201, also pertain to the wound closure device 203. The wound closure device 500 includes a connector 502 that couples two or more of the pads (e.g., the first pad and the second pad 202, 232), or two or more of the entire wound closure devices 200, 201, 203 together. The connector 502 can couple, for example, all of the pads together that are within each of the wound closure devices 200, 201, 203, and can be situated around the periphery of the pads within each wound closure device 200, 201, 203, or around the periphery of the wound closure devices 200, 201, 203. In this case, when the adhesive surfaces of the wound closure devices 200, 201, 203 are exposed (e.g., by removing an adhesive backing), they remain affixed and constrained together. Then, when the system is placed across the wound line 268, the connector 502 can be removed, which can both individualize the movement of each wound closure device 200, 201, 203, and can also allow individualized tensioning of each individual wound closure device 200, 201, 203. Another advantage of the connector 502, is the ability to have predetermined spacing between each individual wound closure device 200, 201, 203, when the wound closure device 500 is placed across the wound line 268, adhered to the individual, and the connector 502 is removed. In some cases, this can be desirable for uniform tensioning, or areas with curvature that may need additional areas of tensioning (e.g., a joint, a knee, an elbow, etc.). In other cases, the individualized and unconstrained movement of each wound closure device 200, 201, 203, allows the wound closure devices 200, 201, 203 to move and respond to localized regions of skin stretching. For example, during flexion, particular regions of skin within the skin region defined by a joint stretches in different magnitudes, particularly depending on the localized curvature.

As illustrated in FIGS. 5A and 5B, the wound closure device 500 can include a second connector 504, where the first connector 502 couples at least the first and the third pads 202, 232 together, and the second connector 504 couples the second and the fourth pads 204, 234 together. In other embodiments, the first connector 502 can couple together a portion of each of the wound closure devices 200, 201, 203, and the second connector 504 can couple together a portion of each of the wound closure devices 200, 201, 203. This can be advantageous, allowing a side of pads (e.g., the first and the third pads 202, 232) to be installed and adhered to one side of the wound line 268. Then when the side of pads (e.g., the first and the third pads 202, 232) is adhered, the first connector 502 can be removed. Then, the opposing sides of pads (e.g., the second and the fourth pads 204, 234) can be adhered to the opposite side of the wound line 268. After, the second connector 504 can be removed. In some cases, the connectors 502, 504 as described above, do not contact the adhesive surfaces of the pads, which can aid in the installation of the wound closure device 500 (e.g., via an easy removal of the connectors 502, 504). In some embodiments, the connectors 502, 504 as described above, can be a film, and the connectors 502, 504 can be connected to the top surfaces of the pads (e.g., the first pad, the second pad, the third pad, the fourth pad 202, 204, 232, 234, respectively). The connectors 502, 504 allow adjacent pads (e.g., the first and the third pads 202, 232) to be attached to each other, thus resulting in a contiguous device (e.g., FIG. 5A). The one or more connectors 502, 504 (e.g., which can be film) are removed after adhering the pads within the wound closure devices 200, 201, 203 to either side of the wound. Thereafter, adjacent sets of pads (e.g., the first and the third pads 202, 232) can move independent of each other in response to elongation/stretching of skin parallel to the wound line 268.

Use of the prior-art device around joints such as the knee, hip, shoulder, elbow etc. poses unique challenges due to the relatively large amount of skin movement occurring during normal activities performed by the patient. When the knee joint flexes, the skin surrounding the incision undergoes significant elongation parallel to the incision. If the wound-closure device does not permit the skin elongation or restricts it, then the range of joint movement may be limited or the attachment of the device to skin maybe compromised. In prior-art device of FIGS. 1A-1B, the ability of the device to allow for such skin elongation is limited by the elasticity of the wound-edge pads. In one embodiment of the present invention, the ability of the device to stretch or elongate together with the skin, is facilitated by the addition of slits and/or perforations on the wound pads (FIGS. 3B, 4A). In another embodiment of the invention, the ability of the device to stretch or elongate parallel to the wound line is facilitated by use of reduced thickness zone/s between portions of the wound pads or between consecutive sets of wound pads (FIG. 4B). Yet another embodiment of the device is composed of two of more consecutive sets of wound pads, with a film adhering to the top of the pads. The film serves to keep adjacent pads attached to each other resulting in a contiguous device (FIG. 5A). This film is removed after adhering the wound-edge pads to either side of the wound. Thereafter, adjacent sets of pads can move independent of each other in response to elongation/stretching of skin parallel to the wound-line (see FIG. 5B).

Another challenge with use of prior-art devices around joints is increased forces experienced by the wound-closure device. As the joint flexes, the skin elongates not only parallel to the wound but also perpendicular to it. This results in increased forces acting across the wound line, which can compromise wound closure. To address this, in one set of embodiments of the invention an alternating connection is provided between the elongate connecting elements via the use of crossbars.

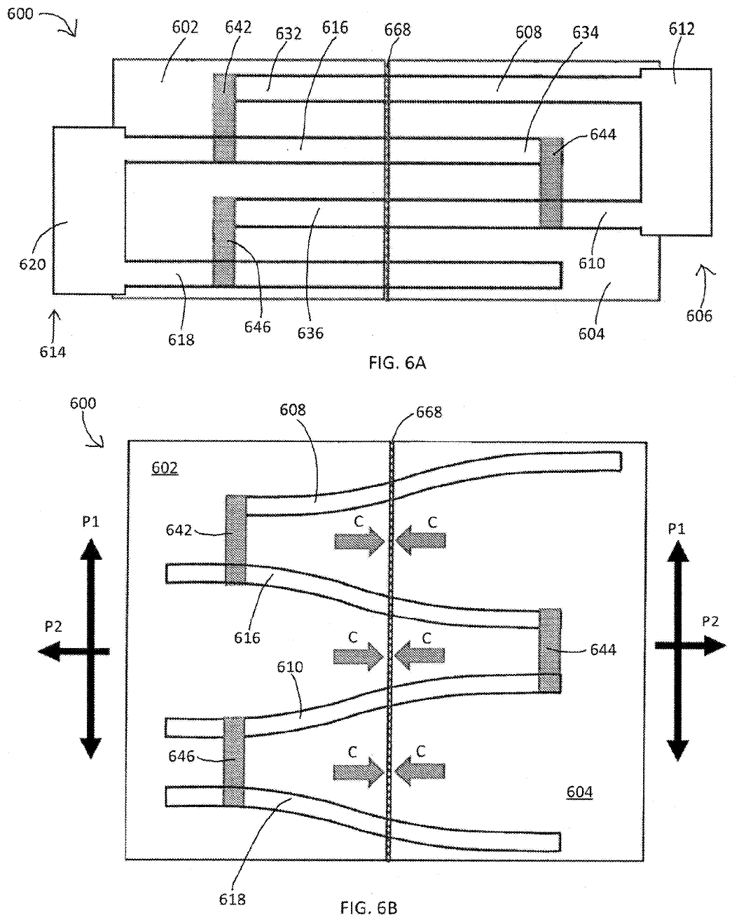

Looking at FIGS. 6A and 6B, there is shown a wound closure device 600 that includes a first pad 602 having an adhesive disposed on an undersurface of the first pad 602, and a second pad 604 having an adhesive disposed on an undersurface of the second pad 604. Emanating from the first pad 602 is a first tension adjusting pad 606 having elongated and generally parallel connecting elements 608, 610 integrally formed with a first section 612. Similarly, emanating from the second pad 604 is a second tension adjusting pad 614 having elongated connecting elements 616, 618 integrally formed with a second section 620.

At a distal end 632 of the connecting element 608 of the first tension adjusting pad 606, there is connected a first crossbar 642. At a distal end 634 of the connecting element 616 of the second tension adjusting pad 614, there is connected a second crossbar 644. At a distal end 636 of the connecting element 610 of the first tension adjusting pad 606, there is connected a third crossbar 646.

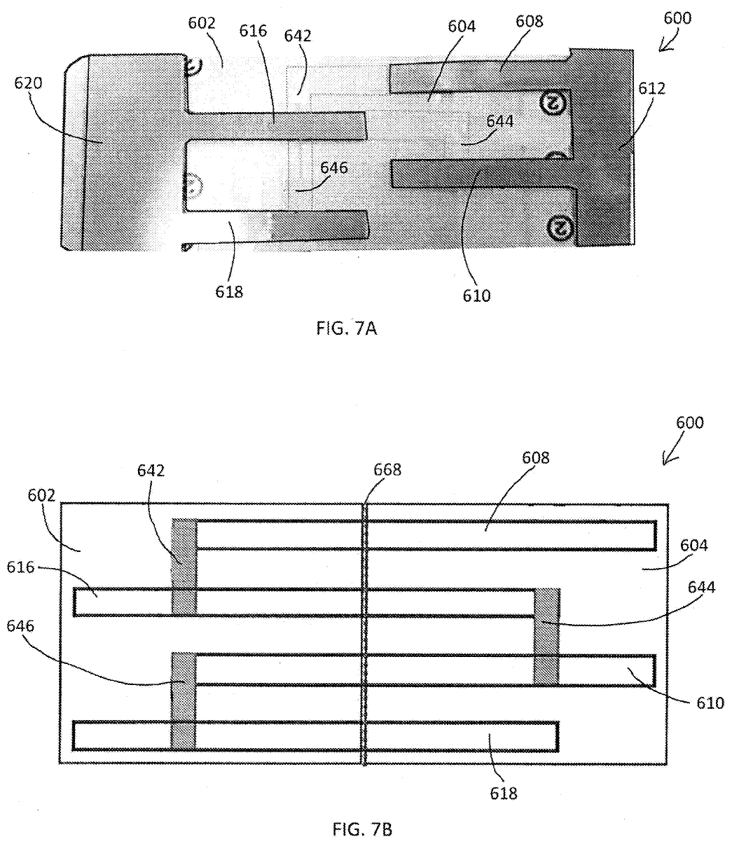

FIGS. 7A and 7B show a sequence of steps resulting in the formation of an alternating connection pattern between the connecting elements 608, 610, 616, 618 and crossbars 642, 644, 646 at the completion of application of the device 600. A first step includes peeling-off a release liner on the adhesive disposed on the undersurface of the first pad 602 and the second pad 604 thereby exposing a portion of the adhesive on the undersurface of the first pad 602 and the second pad 604. Peeling off the release liner results in separation of the first pad 602 and its connected first tension adjusting pad 606 and the second pad 604 and its associated second tension adjusting pad 614. Using the exposed portions of the adhesive of the first pad 602 and the second pad 604, the first pad 602 and the second pad 604 are then individually adhered onto the patient's skin on either side of the wound/incision 668. The second step includes peeling-off a release liner on the adhesive disposed on the undersurface of the first tension adjusting pad 606 and the second tension adjusting pad 614. The third step includes the user lifting the first tension adjusting pad 606 and the second tension adjusting pad 614 away from the patient's skin. Step three includes the user pulling on the first tension adjusting pad 606 and the second tension adjusting pad 614 in a direction away from the wound 668 to close the wound 668. While maintaining this tensile force, the user lays down the first tension adjusting pad 606 and the second tension adjusting pad 614 causing the adhesive on the elongate connecting elements 608, 610, 616, 618 to adhere to the top surface of the first pad 602 and the second pad 604. The first crossbar 642 is trapped underneath the connecting element 616. The second crossbar 644 is trapped underneath the connecting element 610. The third crossbar 646 is trapped underneath the connecting element 618. The next step includes the first section 612 and the second section 620 being snapped-off from the elongate connecting elements 608, 610, 616, 618 (see FIG. 7B).

Looking at FIG. 6B, when the joint flexes, causing the device 600 to stretch parallel P1 and perpendicular P2 to the wound 668, this arrangement of the connecting elements 608, 610, 616, 618 and crossbars 642, 644, 646 causes the device 600 to apply gentle compressive forces C across-the wound 668.

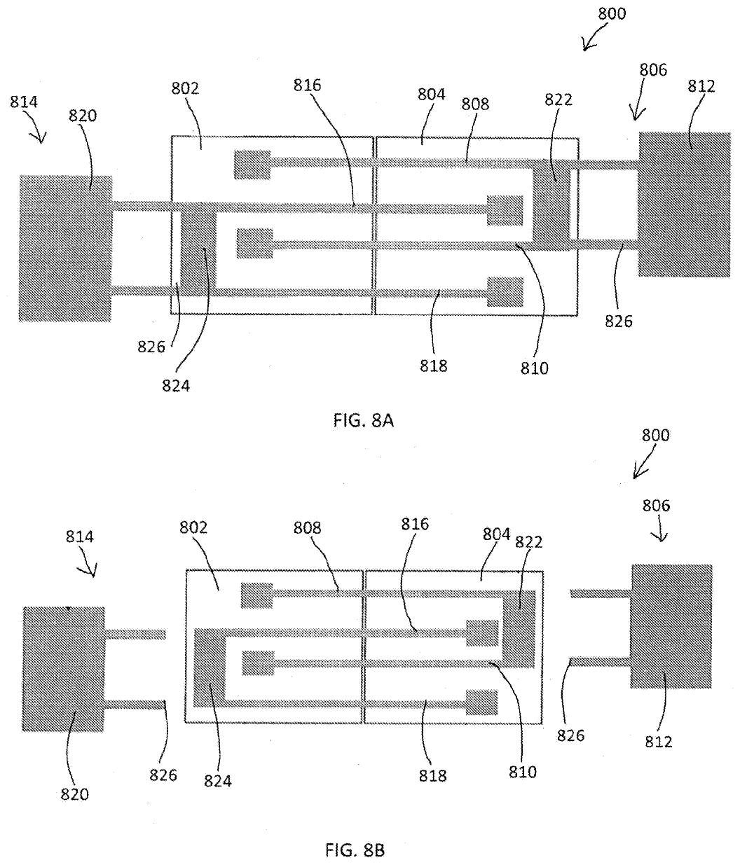

Referring now to FIGS. 8A and 8B, there is shown a wound closure device 800 according to another embodiment. The wound closure device 800 can include a first pad 802, a second pad 804, and a tension adjusting pad 806. The first pad 802 includes a line of material weakness 826 at an outer edge of the first pad 802 extending the first pad 802 in a direction substantially perpendicular to the first tension adjusting pad 806.

The first tension adjusting pad 806 can further include a first section 812 of the first tension adjusting pad 806. The first section 812 can be removably coupled to the first tension adjusting pad 806 at a line of material weakness 826. The first section 812 can be separated from the first tension adjusting pad 806 at the line of material weakness 826. The line of material weakness 826 is a perforation, slit or tapered edge. FIG. 8A shows the first section 812 as it remains integrally connected to the first tension adjusting pad 806. The first section 812 can be removed or separated from the first tension adjusting pad 806 at the line of material weakness 826, as shown in FIG. 8B.

The wound closure device 800 can also include a first connecting element 808, a second connecting element 810, and a first crossbar 822. The first connecting element 808 can extend away from the first section 812 of the first tension adjusting pad 806. The first connecting element 808 is coupled to the first pad 802. The first connecting element 808 can have an adhesive disposed on a surface of the first connecting element 808 to secure the first connecting element 808 to the second pad 804.

The second connecting element 810 can also extend away from the first section 812 of the first tension adjusting pad 806. The second connecting element 810 can be coupled to the first pad 802. The second connecting element 810 can have an adhesive located on a surface of the second connecting element 810 for securing the second connecting element 810 to the second pad 804. The first connecting element 808 and the second connecting element 810 are made of rigidly flexible material.

The first crossbar 822 can have an adhesive located on a surface of the first crossbar 822 connecting the first connecting element 808 to the second connecting element 810.

The wound closure device 800 can further include a second tension adjusting pad 814 coupled to the second pad 802. The second tension adjusting pad 814 can be configured to move the second pad 804 towards the first pad 802 when the second tension adjusting pad 814 is moved away the second pad 804. The second tension adjusting pad 814 can have an adhesive disposed on a surface of the second tension adjusting pad 814 to secure the second tension adjusting pad 814 to the first pad 802. The second tension adjusting pad 814 also has a second section 820 of the second tension adjusting pad 814, a third connecting element 816 and a fourth connecting element 818, both extending away from the second section 820. The second section 820 of the second tension adjusting pad 814 is also removably coupled to the second tension adjusting pad 814 at a line of material weakness 826, similarly to the first section 812 of the first tension adjusting pad 806. The third connecting element 816 can be coupled to the second pad 802 and have an adhesive disposed on a surface of the third connecting element 816 to secure the third connecting element 816 to the first pad 802. The fourth connecting element 818 can also be coupled to the second pad 804 and have an adhesive located on a surface of the fourth connecting element 818 for securing the fourth connecting element 818 to the first pad 802. The third connecting element 816 and the fourth connecting element 818 are made of a rigidly flexible material. The third connecting element 816 and the fourth connecting element 818 can be connected by a second crossbar 824. The second crossbar 824 also has an adhesive disposed on a surface of the second crossbar 824.

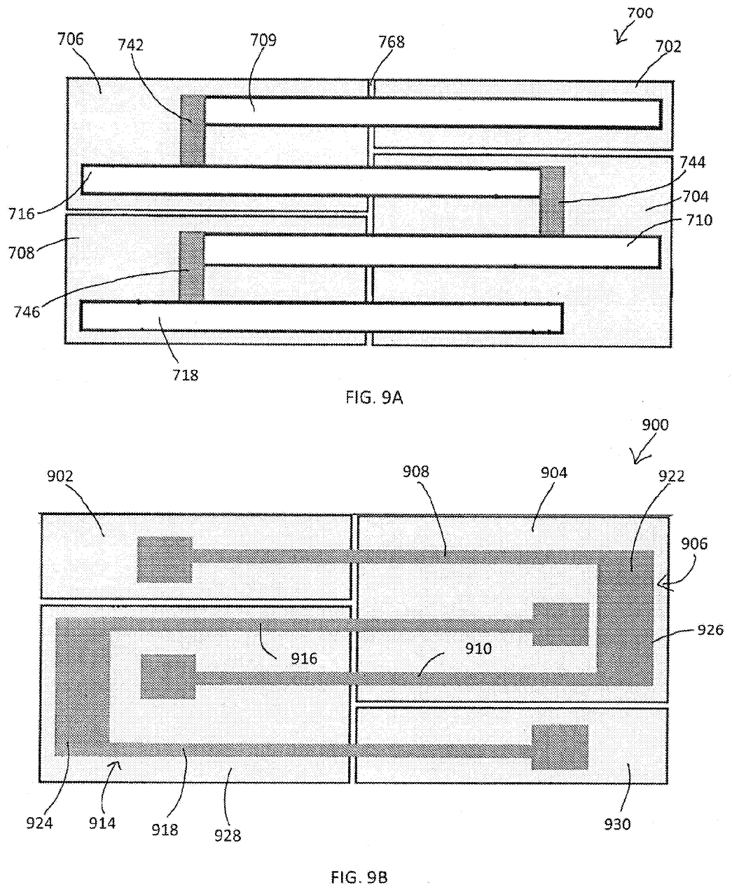

Turning now to FIG. 9A, the wound pads may be composed of multiple segments to facilitate stretching of the wound closure device parallel to the wound line. The example wound closure device 700 includes a first pad 702 having an adhesive disposed on an undersurface of the first pad 702, a second pad 704 having an adhesive disposed on an undersurface of the second pad 704, a third pad 706 having an adhesive disposed on an undersurface of the third pad 706, and a fourth pad 708 having an adhesive disposed on an undersurface of the fourth pad 708. The elongate connecting element 709 is adhered to the top surface of the first pad 702; the elongate connecting element 716 is adhered to the top surface of the third pad 706; the elongate connecting element 710 is adhered to the top surface of the second pad 704, and the elongate connecting element 718 is adhered to the top surface of the fourth pad 708 in the manner described above for FIG. 7. The first crossbar 742 is integral with the connecting element 709 and is trapped underneath the connecting element 716 in the manner described above for FIG. 7. The second crossbar 744 is integral with the connecting element 716 and is trapped underneath the connecting element 710 in the manner described above for FIG. 7. The third crossbar 746 is integral with the connecting element 710 and is trapped underneath the connecting element 718 in the manner described above for FIG. 7. Similar to the wound closure device 600 of FIG. 6A, the wound closure device 700 includes connecting elements 709, 710, 716, 718 and crossbars 742, 744, 746 that cause the device 700 to apply gentle compressive forces across-the wound 768.

Referring now to FIG. 9B, there is shown a wound closure device 900 according to another embodiment. The wound closure device 900 is substantially similar to the wound closure device 800 of FIGS. 8A and 8B. The wound closure device 900 can include a first pad 902 a second pad 904, a third pad 928, and a first tension adjusting pad 906.

The first tension adjusting pad 906 can further include a first section 912 of the first tension adjusting pad 906. The first section 912 can be removably coupled to the first tension adjusting pad 906 at a line of material weakness 926. The first section 912 can be separated from the first tension adjusting pad 906 at the line of material weakness 926. The line of material weakness 926 is a perforation, slit or tapered edge.

The wound closure device 900 can also include a first connecting element 908, a second connecting element 910, and a first crossbar 922. The first connecting element 908 can extend away from the first section 912 of the first tension adjusting pad 906. The first connecting element 908 is coupled to the first pad 902. The first connecting element 906 can have an adhesive disposed on a surface of the first connecting element 906 to secure the first connecting element 908 to the second pad 904.

The second connecting element 910 can also extend away from the first section 912 of the first tension adjusting pad 906. The second connecting element 910 can be coupled to the third pad 928. The second connecting element 910 can have an adhesive located on a surface of the first connecting element 908 for securing the second connecting element 910 to the second pad 904. The first connecting element 908 and the second connecting element 910 are made of rigidly flexible material.

The first crossbar 922 can have an adhesive located on a surface of the first crossbar 928 connecting the first connecting element 908 to the second connecting element 910. The adhesive disposed on the surface of the first crossbar 922 of the first connecting element 908 secures the first crossbar 922 to the second pad 904.

The wound closure device 900 can further include a fourth pad 930, a second tension adjusting pad 914 coupled to the second pad 904. The fourth pad 930 can have an adhesive located on a surface of the fourth pad 930. The second tension adjusting pad 914 can be configured to move the second pad 904 towards the first pad 902 when the second tension adjusting pad 914 is moved away the second pad 904. The second tension adjusting pad 914 can have an adhesive disposed on a surface of the second tension adjusting pad 914 to secure the second tension adjusting pad 914 to the first pad 902. The second tension adjusting pad 914 also has a second section 920 of the second tension adjusting pad 914, a third connecting element 916 and a fourth connecting element 918. The second section 920 of the second tension adjusting pad 914 is also removably coupled to the second tension adjusting pad 914 at a line of material weakness 926, similarly to the first section 912 of the first tension adjusting pad 906. The third connecting element 916 and the fourth connecting element 918 both extend away from the second section 920 of the second tension adjusting pad 914. The third connecting element 916 can be coupled to the second pad 902 and have an adhesive disposed on a surface of the third connecting element 914 to secure the third connecting element 914 to the third pad 928. The fourth connecting element 918 can also be coupled to the fourth pad 928 and have an adhesive located on a surface of the fourth connecting element 918 for securing the fourth connecting element 918 to the third pad 928. The third connecting element 916 and the fourth connecting element 918 are made of a rigidly flexible material. The third connecting element 916 and the fourth connecting element 918 can be connected by a second crossbar 924. The second crossbar 924 also has an adhesive disposed on a surface of the second crossbar 924. The adhesive secures the second crossbar 924 to the third pad 928.

Thus, the invention provides improved wound closure devices that allow for better manipulation and control of the device while closing wounds located around joints such as the knee, hip, shoulder, and elbow.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A wound closure device comprising:
a first pad having an adhesive disposed on a surface of the first pad;
a second pad having an adhesive disposed on a surface of the second pad;
a first tension adjusting pad including a first section, a first connecting element extending away from the first section and a second connecting element extending away from the first section, the first connecting element and the second connecting element being coupled to the first pad, the first tension adjusting pad configured to move the first pad towards the second pad when the first tension adjusting pad is moved away from the first pad, the first connecting element having an adhesive disposed on a surface of the first connecting element for securing the first connecting element to the second pad, the second connecting element having an adhesive disposed on a surface of the second connecting element for securing the second connecting element to the second pad;
a second tension adjusting pad including a second section, a third connecting element extending away from the second section and a fourth connecting element extending away from the second section, the third connecting element and the fourth connecting element being coupled to the second pad, the second tension adjusting pad configured to move the second pad towards the first pad when the second tension adjusting pad is moved away from the second pad, the third connecting element having an adhesive disposed on a surface of the third connecting element for securing the third connecting element to the first pad, the fourth connecting element having an adhesive disposed on a surface of the fourth connecting element for securing the fourth connecting element to the first pad; and
a first crossbar having an adhesive disposed on a surface of the first crossbar for securing the first connecting element to the third connecting element.

2. The wound closure device of claim 1, further comprising:
a second crossbar having an adhesive disposed on a surface of the second crossbar for securing the second connecting element to the third connecting element.

3. The wound closure device of claim 2, further comprising:
a third crossbar having an adhesive disposed on a surface of the third crossbar for securing the second connecting element to the fourth connecting element.

4. A wound closure device comprising:
a first pad having an adhesive disposed on a surface of the first pad;
a second pad having an adhesive disposed on a surface of the second pad;
a third pad having an adhesive disposed on a surface of the third pad;
a fourth pad having an adhesive disposed on a surface of the fourth pad; and
a first tension adjusting pad including a first section, a first connecting element extending away from the first section and a second connecting element extending away from the first section, the first connecting element being coupled to the third pad, the second connecting element being coupled to the fourth pad, the first tension adjusting pad configured to move the third pad and the fourth pad towards the first pad and the second pad when the first tension adjusting pad is moved away from the third pad and the fourth pad, the first connecting element having an adhesive disposed on a surface of the first connecting element for securing the first connecting element to the first pad, the second connecting element having an adhesive disposed on a surface of the second connecting element for securing the second connecting element to the second pad.

5. The wound closure device of claim 4, further comprising:
a second tension adjusting pad including a second section, a third connecting element extending away from the second section and a fourth connecting element extending away from the second section, the third connecting element being coupled to the second pad and the fourth connecting element being coupled to the second pad, the second tension adjusting pad configured to move the second pad towards the third pad and the fourth pad when the second tension adjusting pad is moved away from the second pad, the third connecting element having an adhesive disposed on a surface of the third connecting element for securing the third connecting element to the third pad, the fourth connecting element having an adhesive disposed on a surface of the fourth connecting element for securing the fourth connecting element to the fourth pad.

6. The wound closure device of claim 5, further comprising:
a first crossbar having an adhesive disposed on a surface of the first crossbar for securing the first connecting element to the third connecting element.

7. The wound closure device of claim 6, further comprising:
a second crossbar having an adhesive disposed on a surface of the second crossbar for securing the second connecting element to the third connecting element.

8. The wound closure device of claim 7, further comprising:
a third crossbar having an adhesive disposed on a surface of the third crossbar for securing the second connecting element to the fourth connecting element.

9. A wound closure device comprising:
a first pad having an adhesive disposed on a surface of the first pad;
a second pad having an adhesive disposed on a surface of the second pad;
a first tension adjusting pad coupled to the first pad, the first tension adjusting pad configured to move the first pad towards the second pad when the first tension adjusting pad is moved away from the first pad, the first tension adjusting pad having an adhesive disposed on a surface of the first tension adjusting pad for securing the first tension adjusting pad to the second pad; and
a flexible tab coupled to the first and the second pad, the flexible tab being free of an adhesive.

10. The wound closure device of claim 9, wherein a longitudinal dimension of the flexible tab is positioned on an end of the first pad.

11. The wound closure device of claim 9, wherein a longitudinal dimension of the flexible tab is positioned on an end of the second pad.

12. The wound closure device of claim 9, further comprising a second flexible tab coupled to the first and the second pad, the flexible tab being free of an adhesive.

13. The wound closure device of claim 12, further comprising a third flexible tab coupled to the first and second pad, the third flexible tab being free of an adhesive.

14. The wound closure device of claim 12, wherein the flexible tab and the second flexible tab do not overlap.

15. The wound closure device of claim 12, wherein the flexible tab and the second flexible tab are substantially parallel to each other.

16. The wound closure device of claim 12, the flexible tab and the second flexible comprise a polymeric material.

17. The wound closure device of claim 165, wherein the polymeric material comprises a plastic.

18. The wound closure device of claim 9, wherein the flexible tab is located below the first pad, the second pad, and the first tension adjusting pad.

19. The wound closure device of claim 9, wherein the flexible tab is substantially perpendicular to a wound when the first pad and the second pad are positioned on opposite sides of a wound.

20. The wound closure device of claim 9, further comprising:
a second tension adjusting pad coupled to the second pad, the second tension adjusting pad configured to move the second pad towards the first pad when the second tension adjusting pad is moved away the second pad, the second tension adjusting pad having an adhesive disposed on a surface of the second tension adjusting pad for securing the second tension adjusting pad to the first pad. cm 21. A wound closure device comprising:
a first pad having an adhesive disposed on a surface of the first pad;
a second pad having an adhesive disposed on a surface of the second pad;
a first tension adjusting pad coupled to the first pad, the first tension adjusting pad configured to move the first pad towards the second pad when the first tension adjusting pad is moved away the first pad, the first tension adjusting pad having an adhesive disposed on a surface of the first tension adjusting pad for securing the first tension adjusting pad to the second pad;
a third pad having an adhesive disposed on a surface of the first pad;
a fourth pad having an adhesive disposed on a surface of the second pad;
a second tension adjusting pad coupled to the third pad, the second tension adjusting pad configured to move the third pad towards the fourth pad when the second tension adjusting pad is moved away the third pad, the second tension adjusting pad having an adhesive disposed on a surface of the second tension adjusting pad for securing the second tension adjusting pad to the fourth pad; and
a connector coupled to the first and the third pad.

22. The wound closure device of claim 21, wherein the connector comprises a flexible material.

23. The wound closure device of claim 22, wherein a longitudinal axis of the flexible tab is substantially parallel to a wound when the first pad and the second pad are positioned on opposite sides of a wound and when the third pad and the fourth pad are positioned on opposite sides of the wound.

24. The wound closure device of claim 22, the connector allows parallel movement of the first and the third pad along axis defined by the wound.

25. The wound closure device of claim 21, further comprising a second connector coupled to the second and the third pad.

26. The wound closure device of claim 25, further comprising
a third connector coupled to the first and the second pad; and
wherein the connector and the third connector do not overlap.

27. The wound closure device of claim 21, wherein the connector fixes and prevents movement of the first pad relative to the third pad.

28. The wound closure device of claim 27, wherein the connector is coupled to peripheral edges of the first pad and the third pad.

29. The wound closure device of claim 27, wherein the connector is further coupled to the second pad and the fourth pad.

30. The wound closure device of claim 29, wherein the connector is coupled to peripheral edges of the first pad, the second pad, the third pad, and the fourth pad.

31. The wound closure device of claim 30, wherein the connector does not contact the adhesive surface of the first pad, the second pad, the third pad, or the fourth pad.

32. The wound closure device of claim 26, further comprising a second connector coupled to the second pad and the fourth pad; and
wherein the second connector fixes and prevents movement of the second pad relative to the fourth pad.

33. A wound closure device comprising:
a first pad having an adhesive disposed on a surface of the first pad;
a second pad having an adhesive disposed on a surface of the second pad;
a first tension adjusting pad coupled to the first pad, the first tension adjusting pad configured to move the first pad towards the second pad when the first tension adjusting pad is moved away the first pad, the first tension adjusting pad having an adhesive disposed on a surface of the first tension adjusting pad for securing the first tension adjusting pad to the second pad; and
wherein the first pad has a line of material weakness.

34. The wound closure device of claim 33, wherein the line of material weakness is a slit on an outer edge of the first pad.

35. The wound closure device of claim 34, wherein the slits elongate the first pad in a direction substantially perpendicular to the first tension adjusting pad.

36. The wound closure device of claim 33, wherein the line of material weakness is a plurality of perforations extending across the first pad.

37. The wound closure device of claim 33, wherein the line of material weakness is a plurality of perforations extending across the second pad.

38. The wound closure device of claim 33, further comprising:
a second tension adjusting pad coupled to the second pad, the second tension adjusting pad configured to move the second pad towards the first pad when the second tension adjusting pad is moved away the second pad, the second tension adjusting pad having an adhesive disposed on a surface of the second tension adjusting pad for securing the second tension adjusting pad to the first pad.

39. A wound closure device comprising:
a first pad having an adhesive disposed on a surface of the first pad;
a second pad having an adhesive disposed on a surface of the second pad;
a first tension adjusting pad coupled to the first pad, the first tension adjusting pad configured to move the first pad towards the second pad when the first tension adjusting pad is moved away the first pad, the first tension adjusting pad having an adhesive disposed on a surface of the first tension adjusting pad for securing the first tension adjusting pad to the second pad;
a first section of the first tension adjusting pad;
a first connecting element extending away from the first section of the first tension adjusting pad, wherein the first connecting element is coupled to the first pad, the first connecting element having an adhesive disposed on a surface of the first connecting element for securing the first connecting element to the second pad;
a second connecting element extending away from the first section of the first tension adjusting pad, wherein the second connecting element is coupled to the first pad, the second connecting element having an adhesive disposed on a surface of the first connecting element for securing the second connecting element to the second pad; and
a first crossbar having an adhesive disposed on a surface of the first crossbar connecting the first connecting element to the second connecting element.

40. The wound closure device of claim 39, wherein the first section is removably coupled to the first tension adjusting pad at a line of material weakness.

41. The wound closure device of claim 40, wherein the line of material weakness is a perforation.

42. The wound closure device of claim 39, wherein the first section is separated from the first tension adjusting pad at the line of material weakness.

43. The wound closure device of claim 39, wherein the first connecting element and the second connecting element are rigidly flexible.

44. The wound closure device of claim 39, wherein the first pad includes a line of material weakness at an outer edge of the first pad extending the first pad in a direction substantially perpendicular to the first tension adjusting pad.

45. The wound closure device of claim 44, wherein the line of material weakness is a slit or a perforation.

46. The wound closure device of claim 39, further comprising:
a second tension adjusting pad coupled to the second pad, the second tension adjusting pad configured to move the second pad towards the first pad when the second tension adjusting pad is moved away the second pad, the second tension adjusting pad having an adhesive disposed on a surface of the second tension adjusting pad for securing the second tension adjusting pad to the first pad.

47. The wound closure device of claim 46, further comprising:
a second section of the second tension adjusting pad;
a third connecting element extending away from the second section of the second tension adjusting pad, wherein the third connecting element is coupled to the second pad, the third connecting element having an adhesive disposed on a surface of the third connecting element for securing the third connecting element to the first pad;
a fourth connecting element extending away from the second section of the second tension adjusting pad, wherein the fourth connecting element is coupled to the second pad, the fourth connecting element having an adhesive disposed on a surface of the fourth connecting element for securing the fourth connecting element to the first pad; and
a second crossbar having an adhesive disposed on a surface of the second crossbar connecting the third connecting element to the fourth connecting element.

48. The wound closure device of claim 47, wherein the second section of the second tension adjusting pad is removably coupled to the second tension adjusting pad at a line of material weakness.

49. The wound closure device of claim 47, wherein the third connecting element and the fourth connecting element are rigidly flexible.

50. A wound closure device comprising:
a first pad having an adhesive disposed on a surface of the first pad;
a second pad having an adhesive disposed on a surface of the second pad;
a third pad having an adhesive disposed on a surface of the third pad;
a first tension adjusting pad coupled to the first pad, the first tension adjusting pad configured to move the first pad towards the second pad when the first tension adjusting pad is moved away the first pad, the first tension adjusting pad having an adhesive disposed on a surface of the first tension adjusting pad for securing the first tension adjusting pad to the second pad;
a first section of the first tension adjusting pad;
a first connecting element extending away from the first section of the first tension adjusting pad, wherein the first connecting element is coupled to the first pad, the first connecting element having an adhesive disposed on a surface of the first connecting element for securing the first connecting element to the second pad;
a second connecting element extending away from the first section of the first tension adjusting pad, wherein the second connecting element is coupled to the third pad, the second connecting element having an adhesive disposed on a surface of the first connecting element for securing the second connecting element to the second pad; and
a first crossbar having an adhesive disposed on a surface of the first crossbar connecting the first connecting element to the second connecting element.

51. The wound closure device of claim 50, wherein the adhesive disposed on the surface of the first crossbar of the first connecting element for securing the first crossbar to the second pad.

52. The wound closure device of claim 50, wherein the first section is removably coupled to the second pad at a line of material weakness.

53. The wound closure device of claim 52, wherein the first section is separated from the first tension adjusting pad at the line of material weakness.

54. The wound closure device of claim 52, wherein the line of material weakness is a perforation.

55. The wound closure device of claim 50, wherein the first connecting element and the second connecting element are rigidly flexible.

56. The wound closure device of claim 50, further comprising:
a second tension adjusting pad coupled to the second pad, the second tension adjusting pad configured to move the second pad towards the first pad when the second tension adjusting pad is moved away the second pad, the second tension adjusting pad having an adhesive disposed on a surface of the second tension adjusting pad for securing the second tension adjusting pad to the first pad.

57. The wound closure device of claim 56, further comprising:
a fourth pad having an adhesive disposed on a surface of the fourth pad;
a second section of the second tension adjusting pad;
a third connecting element extending away from the second section of the second tension adjusting pad, wherein the third connecting element is coupled to the second pad, the third connecting element having an adhesive disposed on a surface of the third connecting element for securing the third connecting element to the third pad;
a fourth connecting element extending away from the second section of the second tension adjusting pad, wherein the fourth connecting element is coupled to the fourth pad, the fourth connecting element having an adhesive disposed on a surface of the fourth connecting element for securing the fourth connecting element to the third pad; and
a second crossbar having an adhesive disposed on a surface of the second crossbar connecting the third connecting element to the fourth connecting element, wherein the adhesive secures the second crossbar to the third pad.

58. The wound closure device of claim 57, wherein the second section of the second tension adjusting pad is removably coupled to the second tension adjusting pad at a line of material weakness.

59. The wound closure device of claim 57, wherein the third connecting element and the fourth connecting element are rigidly flexible.
